(19) United States
(12) Patent Application Publication
(10) Pub. No.: US 2004/0106665 A1
(43) Pub. Date: Jun. 3, 2004

(54) Resolution process for preparation of substantially pure (r) and (s) enantiomers of 2-(4-nitroimidazolyl)-4-methodxyphenylpropionic acid and salts thereof

(76) Inventors: Thomas Joseph Kress, Greenwood, IN (US); Roger Lewis Robey, Greenwood, IN (US); James Patrick Wepsiec, Nineveh, IN (US); Charles Arthur Wepsiec, Nineveh, IN (US); Gary Anthony Rhodes, Indianapolis, IN (US)

Correspondence Address: ELI LILLY AND COMPANY, PATENT DIVISION, P.O. BOX 6288, INDIANAPOLIS, IN, 46206-6288, US

(21) Appl. No.: 10/381,164

(22) PCT Filed: Sep. 28, 2001
(86) PCT No.: PCT/US01/27739

(30) Foreign Application Priority Data
Oct. 13, 2000 (US) 60240350

(51) Int. Cl. A61K 31/4172; C07D 233/91

(52) U.S. Cl. 514/398; 548/327.1

(57) ABSTRACT

A method for optically resolving a racemic mixture of 2-(4-nitroimidazolyl)-4-methoxyphenylproprionic acid to substantially pure (R) and (S) enantiomers salts thereof which are useful intermediates in the preparation of certain growth hormone secretagogues.

Growth hormone (GH) is a secretory protein of the pituitary gland of animals having wide ranging developmental effects on the organism. Artificial manipulation of growth hormone levels has been demonstrated to have significant therapeutic utility. Human growth hormone supplementation has been shown to be an effective treatment for growth hormone deficiencies and their related disease states in humans. Studies have uncovered new and significant properties of growth hormone which lend further importance to the ability to control growth hormone levels. For example, recent clinical studies indicate that growth hormone supplementation may be useful in combating the maladies of aging in humans. Elevated growth hormone levels in animals have been shown to result in increased lean muscle mass.

While growth hormone is naturally produced by the pituitary gland, the secretion of growth hormone into the bloodstream is controlled by a second protein, Growth Hormone Releasing Factor (GRF). This hormone is also commonly known in the art as somatocrinin, Growth Hormone Releasing Hormone (GHRH), and Growth Releasing Hormone (GRH). In recent years, significant efforts have been taken to develop nonpeptidyl analogs of a series of compounds identified as growth hormone secretagogues (GHS) which induce the production or release of growth hormone (GH), and act in concert or synergistically with growth hormone releasing hormone (GHRH).

International Patent Application No. PCT/US/17229, which is incorporated in its entirety herein by reference, discloses potent and effective growth hormone secretagogues. Some of these compounds exist as diastereomers due to the presence of two chiral carbon atoms. The separation of these diastereomers can be achieved by chromatographic separation of a diastereomeric intermediate or the final product. The obvious shortcomings of chromatographic separation are inefficiency and expense. The separation is particularly inefficient if the separation is performed as the final step where one-half of the final product may be discarded as the undesired diastereomer.

The present invention provides compounds and salts thereof that are useful as intermediates in the preparation of substantially pure (R)-2-(4-nitroimidazolyl)-4-methoxyphenylpropionic acid and substantially pure (S)-2-(4-nitroimidazolyl)-4-methoxyphenylpropionic acid which are useful in the preparations of growth hormone secretagogues (GHS) and diastereomers thereof. The substantially pure enantiomers of the present invention are prepared by a process generally characterized as an optical resolution in which a mixture of diastereomeric salts may be prepared, and then the two diastereomeric salts may be separated. A commonly employed method for the resolution of the racemic mixture (or mixture of enantiomers) into the individual enantiomers is to first convert the enantiomers to diastereomeric salts by way of forming the salts with an optically active acid or base. These diastereomeric salts may then be separated using differential solubility, fractional crystallization, or the like. Further details regarding resolution of enantiomeric mixtures may be found in Jean Jacques, et al., Enantiomers, Racemates, and Resolutions, (Krieger Pub. Co., Malabar, Fla., 1991).

Unfortunately, when attempting to apply these general teachings to efficiently separate enantiomers, it is impossible to determine what conditions or resolving agents will be successful. In the present invention, many resolving agents and/or solvents and/or combinations of solvents under various conditions produced unacceptable mixtures of diastereoisomeric salts. The present invention, however, provides processes for preparing the compounds of the present invention by disclosing remarkably effective resolving agents and conditions that selectively crystallize the substantially pure diastereomeric salts.

A method is described for obtaining a single enantiomer of nitroimidazolyl methoxyphenylpropionic acid of Formula A:

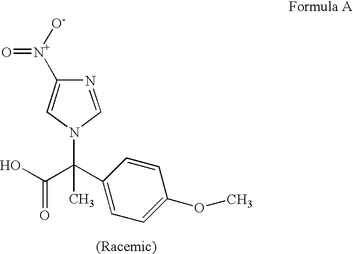

comprising the steps of:

(a) reacting a racemic mixture of a compound of Formula A with a resolving agent selected from the group consisting of levamisole, quinidine, brucine, (+)-cinchonine, (−)-cinchonidine, (1R,2S)-ephedrine and (1S,2R)-ephedrine in a solvent to produce a crystalline salt;

(b) isolating the crystalline salt; and

(c) optionally converting the crystalline salt to free acid thereby producing a substantially pure R or S enantiomer.

The optical resolution method of the instantly claimed invention provides for an optically active form of Formula A to substantially pure enantiomeric forms with (levamisole); by (1S,2R)-ephedrine; by quinidine; by brucine, by (+)-cinchonine, and by (−)-cinchonidine to give (R)-2-(4-nitroimidazolyl)-4-methoxyphenylpropionic acid and by (1R, 2S)-ephedrine, by (+)-cinchonine, by (−)-cinchonidine, and by quinine to give (S)-2-(4-nitroimidazolyl)-4-methoxyphenylpropionic acid. These compounds are useful in the preparation of growth hormone secretagogues (GHS) according to the following Formula I, and are especially useful in obtaining the respective substantially pure diastereomeric growth hormone secretagogues.

The substantially pure R- or S-isomers are also claimed.

The optical resolution method instantly claimed is useful to prepare the (R) and (S) isomers of nitroimidazolyl methoxyphenylpropionic acid which are intermediates useful in the synthesis of growth hormone secretagogues. These isomers can be also used as analytical standards for analysis of the preferred compound which has the (R) stereochemistry.

The preferred growth hormone secretagogue of Formula X

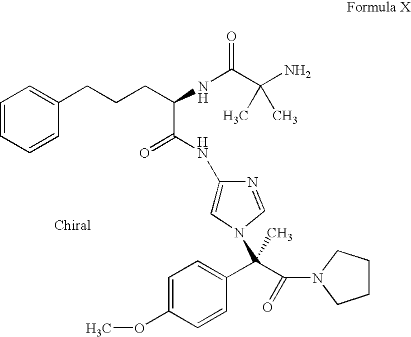

can be prepared utilizing the resolution technology described herein as shown in Scheme I.

As defined herein, the substantially pure R— or S-isomers obtained herein are represented by compounds of Formulae B and C:

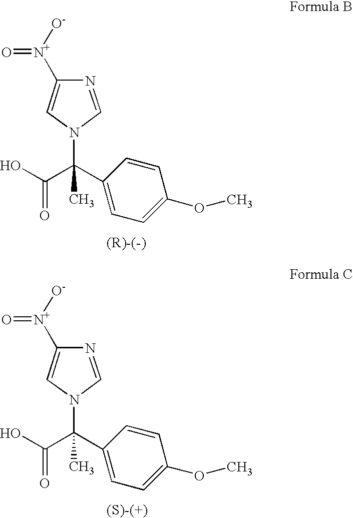

The GHS of Formula X can be prepared using the resolution method described herein as shown in Scheme 1.

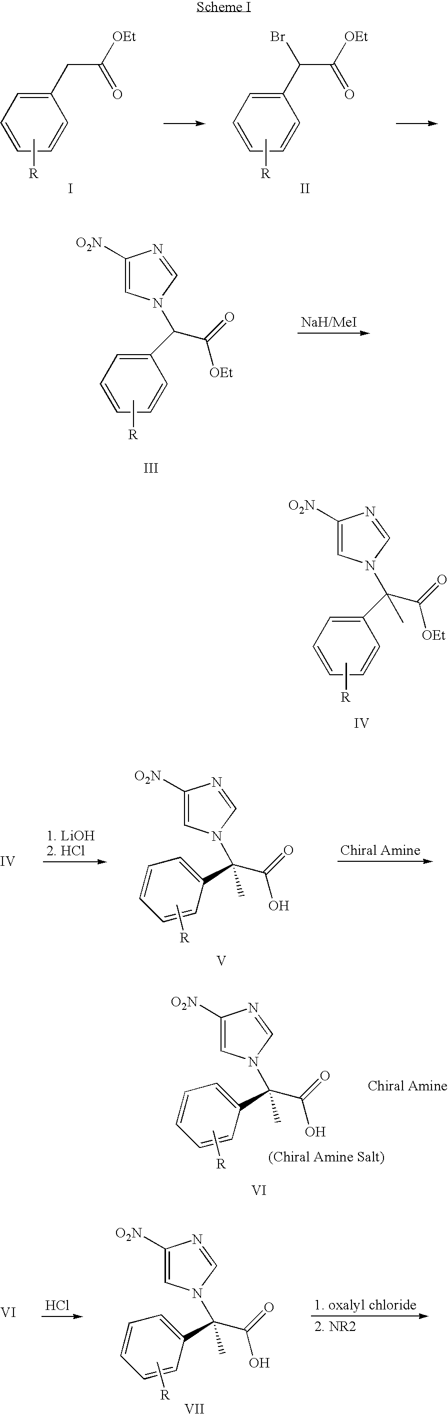

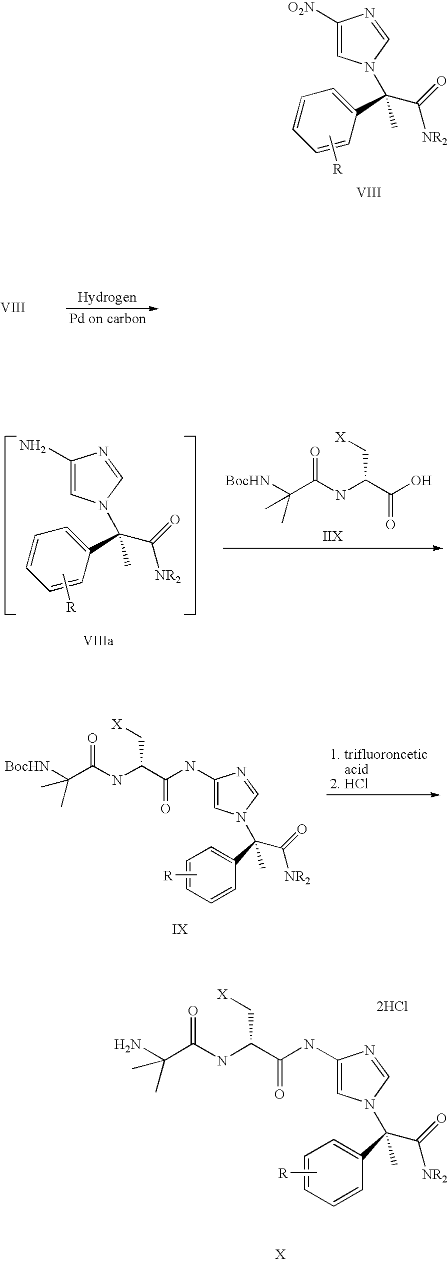

Within Scheme I, wherein R=—OCH₃ (para position); R₂=pyrrolidinyl; X=C₆H₅(CH₂)₂—; and Boc=butoxycarbonyl, a compound of Formula IV may be prepared by the alkylation of a compound of Formula III by standard methods using a base, such as sodium hydride, followed by treatment with an electrophile, such as methyl iodide. Preferred bases for this reaction include sodium, lithium, or potassium hexamethyldisilazide, lithium diisopropylamide, and sodium hydride. Preferred methylating agents include methyl halides or any methyl group with a suitable substituted leaving group such as tosylate, mesylate, and the like.

A compound of Formula V may be prepared by hydrolysis of a compound of Formula IV using standard saponfication conditions known in the art. Suitable reagents for this transformation include sodium hydroxide or lithium hydroxide. As instantly claimed, the resulting carboxylic acid is resolved to give (R) or (S) isomer by reaction with a resolving agent selected from the group consisting of levamisole, (−)-(1R,2S)-ephedrine, (+)-(1S,2R)-ephedrine, quinidine, brucine, (−)-cinchonidine, (+)-cinchonine, or quinine. The diastereomeric salt may be converted to the desired acid VII by standard conditions known in the art. Suitable reagents for this include aqueous hydrochloric or sulfuric acids. The preferred reagent is HCl. Compound VII can be converted to growth hormone secretagogue X by methods described in PCT/US/17229.

Schemes II and III show some of the general procedures for resolution utilized within the present invention. The resolution process of the present invention provides an inexpensive and efficient method for preparing a single enantiomer from racemic nitroimidazolyl methoxyphenyl-propionic acid also referred to as Formula A herein via the formation of a crystalline salt with a resolving agent. The enantiomeric purity of the isomer is from about 90% to about 99%. The solvents used in the resolution step may be varied. The solvents include, but are not limited to methyl t-butyl ether (MTBE), acetone, ethyl acetate/acetone, and acetonitrile. Schemes II and III illustrate the general procedures of the resolution method of the present invention.

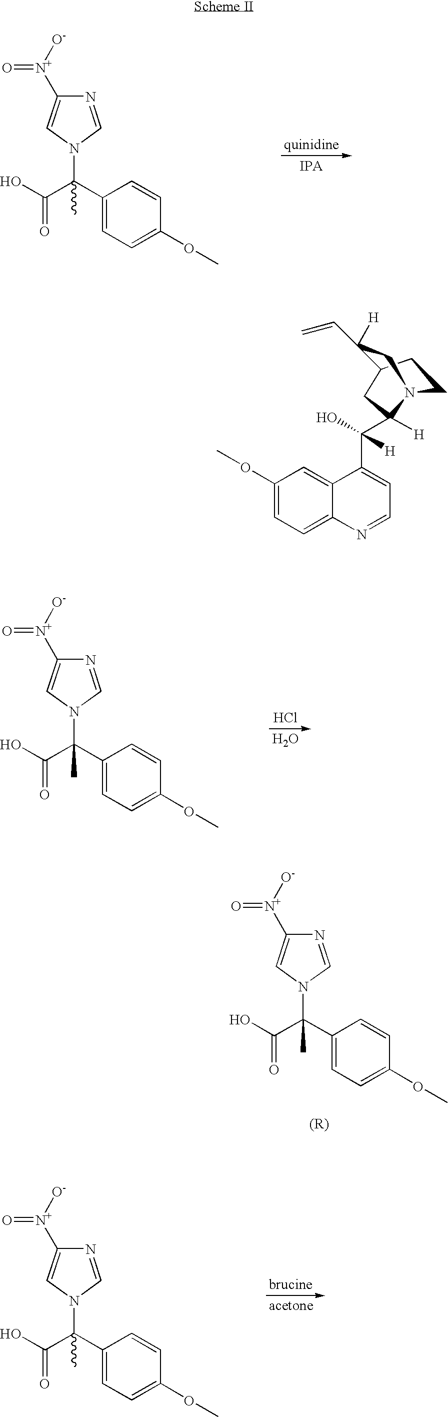

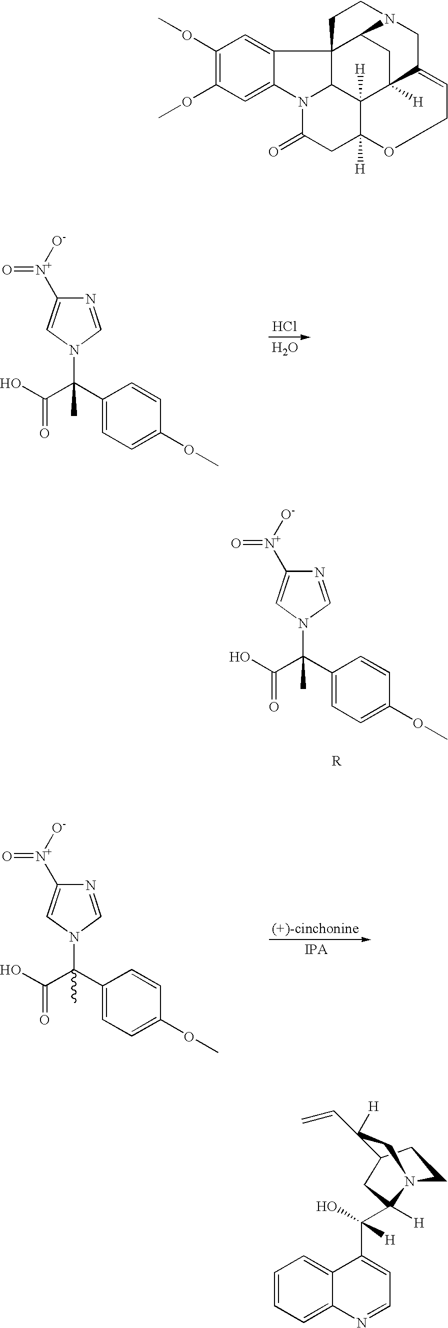

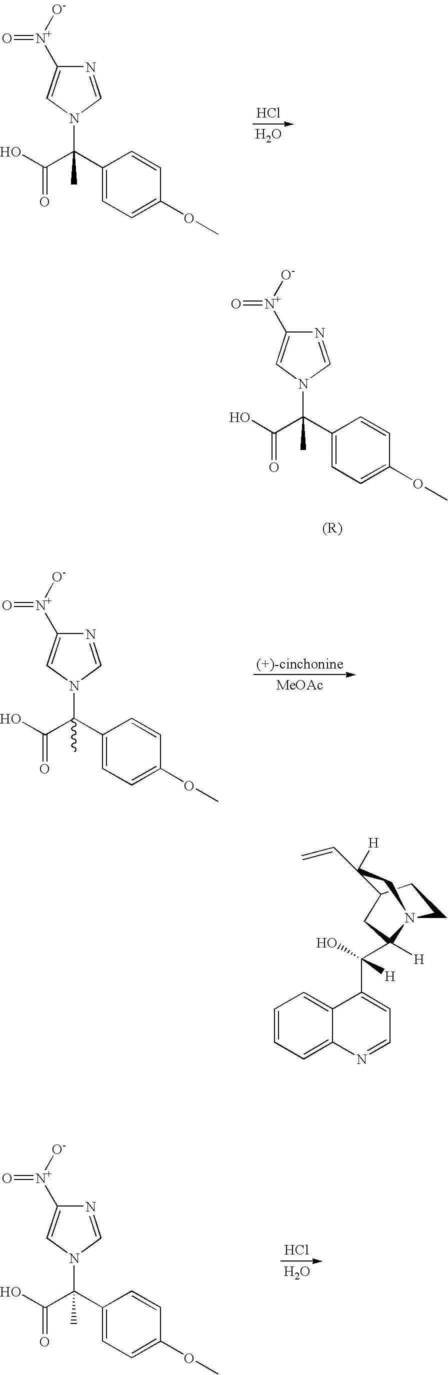

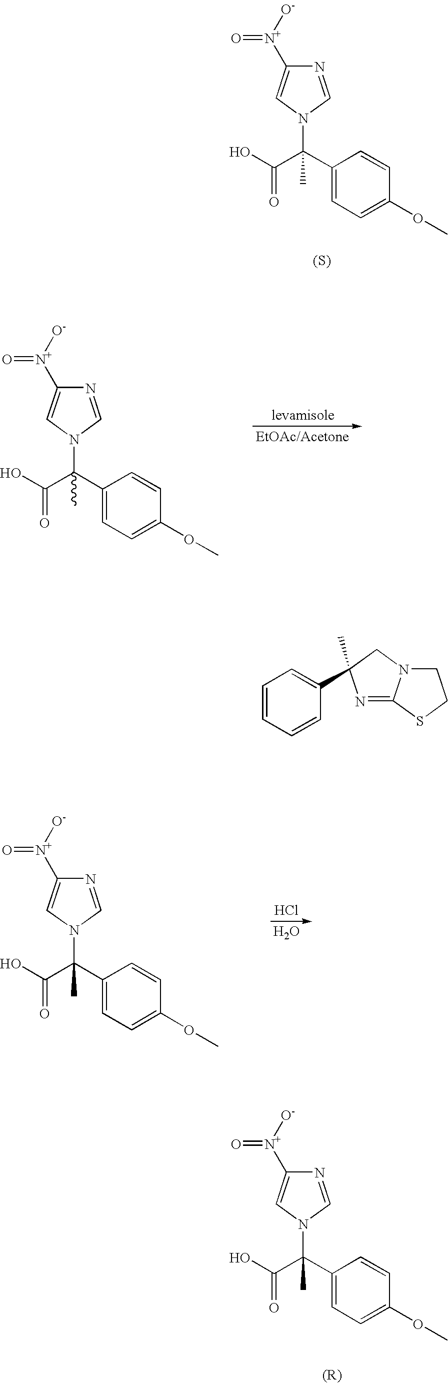

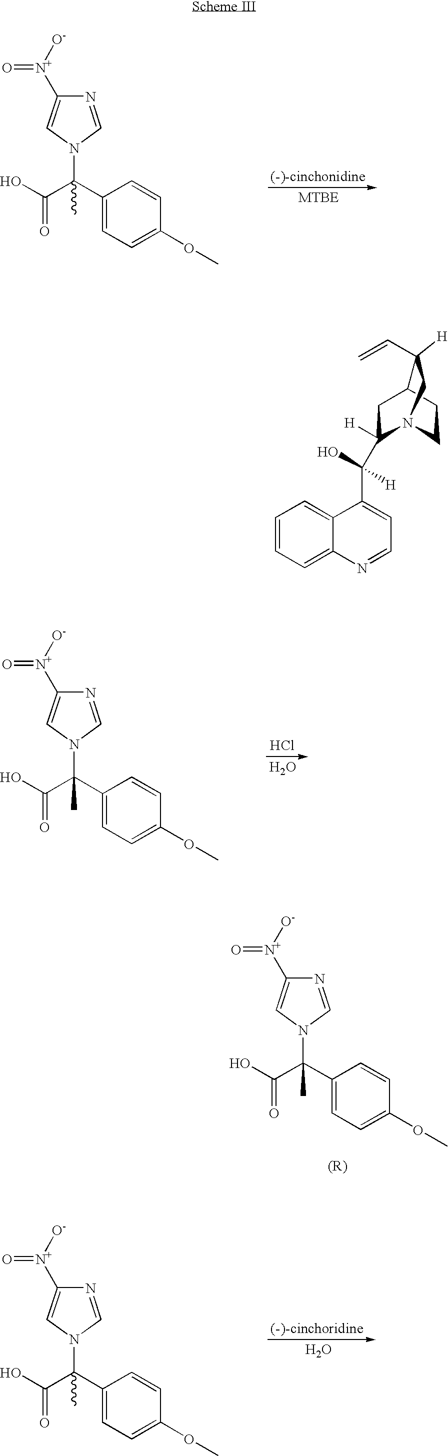

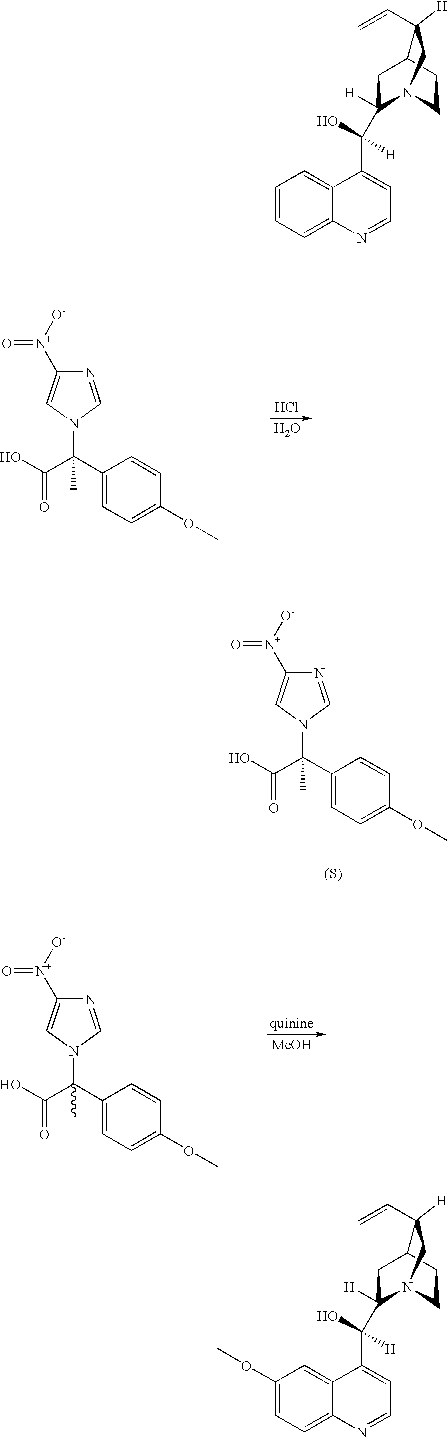

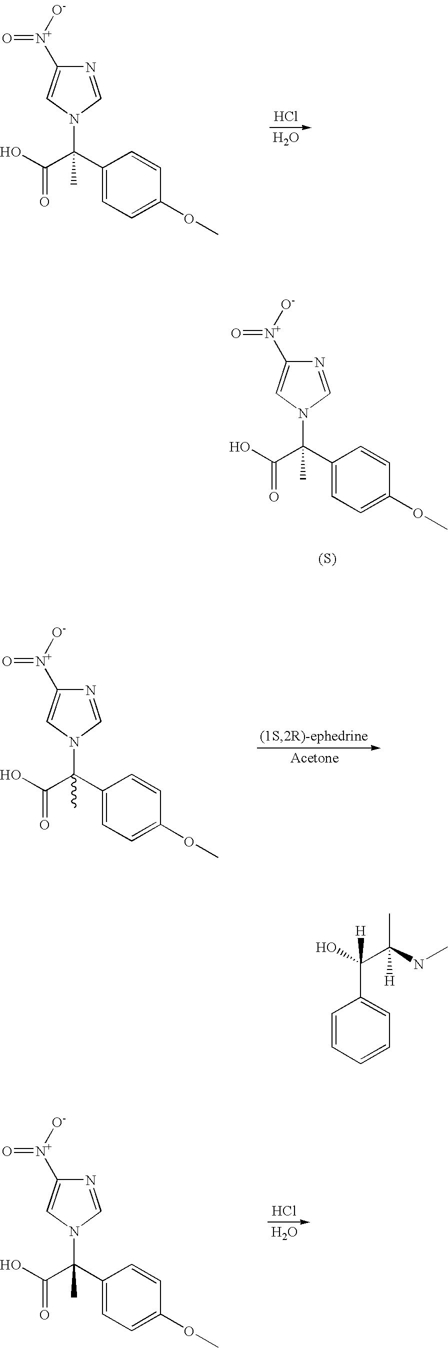

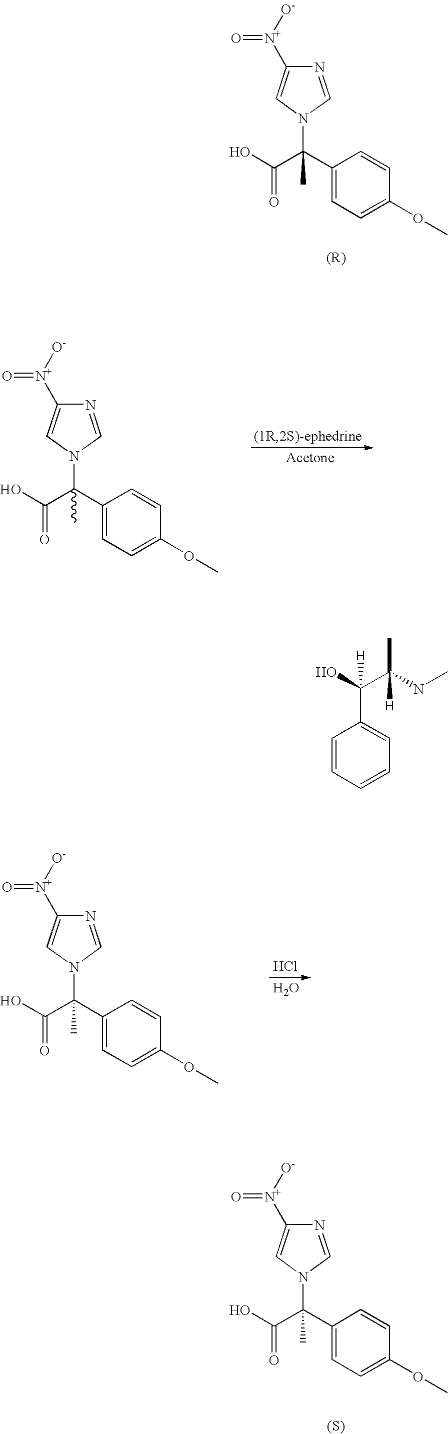

The following examples, and specific preparations therein, are provided to further illustrate the invention.

### EXAMPLE 1

### Preparation of Levamisole Salt of (R)-2-(4-nitroimidazolyl)-4-methoxyphenylproprionic Acid (Ethyl Acetate/Acetone)

A. Preparation of (−)-2, 3, 5, 6-tetrahydro-6-phenylimidazo[2,1-b]Thiazole (Levamisole) from Levamisole Hydrochloride

A mixture of 10.0 g (41.5 mmol) of levamisole hydrochloride (Commercial Source: Aldrich, Catalog Number 19, 614-2), 100 mL of methyl t-butyl ether (MTBE), and 50 mL of 1N NaOH was stirred at 25° C. for 15 min. The layers were separated and the organic layer was washed with brine and dried over sodium sulfate. After filtration to remove the sodium sulfate, the filtrate was concentrated to give 8.14 g (95.9%) of levamisole free base.

B. Levamisole Salt of (R)-2-(4-nitroimidazolyl)-4-methoxyphenylproprionic Acid (Ethyl Acetate/Acetone)

A mixture of the 10 g (34.33 mmol) of racemic 2-(4-methoxyphenyl)-4-nitroimidazolyl)propanoic acid, 7.8 g (38.18 mmol) of levamisole, 150 mL of ethyl acetate and 15 mL of acetone was stirred at reflux to give a yellow solution. The mixture was allowed to cool to 25° C., seeded, and stirred overnight at 25° C. The resulting thick slurry was cooled to 0° C. and filtered and the filter cake was washed twice with cold ethyl acetate. The wet cake was dried in vacuum to give 6.44 g of crude product (37.9% yield).

Using the procedure described above except for changing the solvent to MTBE, the crude salt (1.0 g, 2.0 mmol) was combined with 50 mL of MTBE and 25 mL of 1N HCl and the organic phase separated, washed with brine, and dried over sodium sulfate. After filtration to remove sodium sulfate the filtrate was concentrated to give 0.61 g of R-(−)-2-(4-methoxyphenyl)-4-nitroimidazolyl)propanoic acid (91% ee by chiral capillary electrophoresis).

The crude salt (5.0 g, 10 mmol; giving free acid at 91% ee) was slurried in 75 mL of ethyl acetate at reflux for 1 h, cooled to 25° C., and stirred overnight at 25° C. The slurry was cooled to 0° C. and filtered and the filter cake was washed with cold ethyl acetate and dried to give 4.51 g (90.2% recovery) of purified salt. Conversion of 1.0 g of the salt to the free acid by reaction with 50 mL of MTBE and 25 mL of 1N HCl as above gave 0.64 g of R-(−)-2-(4-methoxyphenyl)-4-nitroimidazolyl)propanoic acid, which was 99.4% ee by chiral capillary electrophoresis.

Enantiomeric excess was determined by chiral capillary electrophoresis using a 75 cm total length capillary/50 cm length to detector×50 μm uncoated capillary; 30° C.; detection at 195 nm; −25 kV; eluent 25 mM sodium phosphate pH 2.5 containing 2,6-dimethyl β-cyclodextrin; injection 0.5 sec×5″; samples at 0.2 mg/mL (10% acetonitrile in water).

### EXAMPLE 2

### Preparation of Quinidine Salt of (R)-(−)-2-(4-methoxyphenyl)-4-nitroimidazolyl)propanoic Acid

A mixture of racemic 2-(4-methoxyphenyl)-4-nitroimidazolyl)propanoic acid (1.02 g, 3.5 mmol) and quinidine (1.14 g, 3.5 mmol) were combined in isopropyl alcohol (20 mL). The resulting slurry was heated to reflux and stirred until all solids dissolved. The resulting yellow solution was allowed to cool to ambient temperature, seeded, and stirred for three days. The thick slurry was filtered, rinsed with isopropyl alcohol (4 mL), and dried to give 0.71 g (33%) of the quinidine salt of (R)-(−)-2-(4-methoxyphenyl)-4-nitroimidazolyl)propanoic acid. The enantiomeric excess was determined directly on the quinidine salt and was found to be 95.5% by chiral capillary electrophoresis using a 72/50 cm×50 μm uncoated capillary; 30° C., detection at 195 nm; −25 kV; eluent 25 mM phosphate pH 2.5+5 mM sulfobutyl ether β-cyclodextrin; injection 0.5 sec×5″; samples at 0.2 mg/mL (10% acetonitrile).

Using the above procedure except using methyl tert butyl ether/ethanol (4:1) instead of isopropyl alcohol gave the quinidine salt of (R)-(−)-2-(4-methoxyphenyl)-4-nitroimidazolyl)propanoic acid in 10% yield with an enantiomeric excess of 94.5%.

### EXAMPLE 3

### Preparation of Brucine Salt of (R)-(−)-2-(4-methoxyphenyl)-4-nitroimidazolyl)propanoic Acid

A mixture of racemic 2-(4-methoxyphenyl)-4-nitroimidazolyl)propanoic acid (0.10 g, 0.3433 mmol) and brucine (Commercial Source: Aldrich, Catalog Number 39, 902-7) (0.136 g, 0.3433 mmol) were combined in acetone (2 mL). The resulting slurry was heated to reflux and stirred until all solids dissolved. The resulting yellow solution was allowed to cool to ambient temperature and stirred overnight. The thick slurry was filtered and dried to give 0.09 g (38%) of the brucine salt of (R)-(−)-2-(4-methoxyphenyl)-4-nitroimidazolyl)propanoic acid, mp 212-214° C. The enantiomeric excess was determined directly on the brucine salt and was found to be 91.3% by chiral capillary electrophoresis using a 72/50 cm×50 μm uncoated capillary; 30° C., detection at 195 nm; −25 kV; eluent 25 mM phosphate pH 2.5+5 mM sulfobutyl ether β-cyclodextrin; injection 0.5 sec×5″; samples at 0.2 mg/mL (10% acetonitrile).

Using the above procedure except using ethyl acetate instead of acetone gave the brucine salt of (R)-(−)-2-(4-methoxyphenyl)-4-nitroimidazolyl)propanoic acid in 34% yield with an enantiomeric excess of 86.6%.

### EXAMPLE 4

### Preparation of (+)-Cinchonine Salt of (R)-(−)-2-(4-methoxyphenyl)-4-nitroimidazolyl)Propanoic Acid

A mixture of racemic 2-(4-methoxyphenyl)-4-nitroimidazolyl)propanoic acid (0.10 g, 0.3433 mmol) and (+)-cinchonine (Commercial Source: Aldrich, Catalog Number 85, 727-0) (0.101 g, 0.3433 mmol) were combined in isopropyl alcohol (2 mL). The resulting slurry was heated to reflux and stirred until all solids dissolved. The resulting yellow solution was allowed to cool to ambient temperature, seeded, and stirred overnight. The thick slurry was filtered and dried to give 0.07 g (35%) of the (+)-cinchonine salt of (R)-(−)-2-(4-methoxyphenyl)-4-nitroimidazolyl)propanoic acid, mp 188-190° C. The enantiomeric excess was determined directly on the (+)-cinchonine salt and was found to be 91.3% by chiral capillary electrophoresis using a 72/50 cm×50 μm uncoated capillary; 30° C., detection at 195 nm; −25 kV; eluent 25 mM phosphate pH 2.5+5 mM sulfobutyl ether O-cyclodextrin; injection 0.5 sec×5″; samples at 0.2 mg/mL (10% acetonitrile).

Using the above procedure except using methyl tert butyl ether/ethanol (2:1) instead of isopropyl alcohol gave the cinchonine salt of (R)-(−)-2-(4-methoxyphenyl)-4-nitroimidazolyl)propanoic acid in 20% yield with an enantiomeric excess of 87.4%.

### EXAMPLE 5

### Preparation of (+)-Cinchonine Salt of (S)-(+)-2-(4-methoxyphenyl)-4-nitroimidazolyl)Propanoic Acid

A mixture of racemic 2-(4-methoxyphenyl)-4-nitroimidazolyl)propanoic acid (0.10 g, 0.3433 mmol) and (+)-cinchonine (Commercial Source: Aldrich, Catalog Number 85, 727-0) (0.101 g, 0.3433 mmol) were combined in methyl acetate (2 mL). The resulting slurry was heated to reflux and stirred until all solids dissolved. The resulting yellow solution was allowed to cool to ambient temperature and stirred overnight. The thick slurry was filtered and dried to give 0.07 g (35%) of the (+)-cinchonine salt of (S)-(+)-2-(4-methoxyphenyl)-4-nitroimidazolyl)propanoic acid. The enantiomeric excess was determined directly on the (+)-cinchonine salt and was found to be 92.7% by chiral capillary electrophoresis using a 72/50 cm×50 μm uncoated capillary; 30° C., detection at 195 nm; −25 kV; eluent 25 mM phosphate pH 2.5+5 mM sulfobutyl ether O-cyclodextrin; injection 0.5 sec×5″; samples at 0.2 mg/mL (10% acetonitrile).

Using the above procedure except using dimethoxyethane instead of methyl acetate gave the (+)-cinchonine salt of (S)-(+)-2-(4-methoxyphenyl)-4-nitroimidazolyl)propanoic acid in 25% yield with an enantiomeric excess of 94.0%.

### EXAMPLE 6

### Preparation of Quinine Salt of (S)-(+)-2-(4-methoxyphenyl)-4-nitroimidazolyl)Propanoic Acid

A mixture of racemic 2-(4-methoxyphenyl)-4-nitroimidazolyl)propanoic acid (0.10 g, 0.3433 mmol) and quinine (Commercial Source: Aldrich, Catalog Number 14, 590-4) (0.11 g, 0.3433 mmol) were combined in methyl alcohol (2 mL). The resulting slurry was heated to reflux and stirred until all solids dissolved. The resulting yellow solution was allowed to cool to ambient temperature and stirred overnight. The thick slurry was filtered and dried to give 0.07 g (33%) of the quinine salt of (S)-(+)-2-(4-methoxyphenyl)-4-nitroimidazolyl)propanoic acid. The enantiomeric excess was determined directly on the quinine salt and was found to be 80.8% by chiral capillary electrophoresis using a 72/50 cm×50 pm uncoated capillary; 30° C., detection at 195 nm; −25 kV; eluent 25 mM phosphate pH 2.5+5 nM sulfobutyl ether β-cyclodextrin; injection 0.5 sec×5″; samples at 0.2 mg/mL (10% acetonitrile).

### EXAMPLE 7

### Preparation of (1S,2R)-(+)-ephedrine Salt of (R)-(−)-2-(4-methoxyphenyl)-(4-nitroimidazolyl)propanoic Acid, Preparation by Recrystallization

To a solution of 1.82 g (11.0 mmol) of (1S,2R)-ephedrine in 9 mL of reagent acetone at 25° C. was added 3.10 g (10.7 mmol) of racemic 2-(4-methoxyphenyl)-(4-nitroimidazolyl)propanoic acid. All solids dissolved and the solution was seeded with a crystal of the title compound. The resulting slurry was stirred overnight at 25° C., diluted with 9 mL of reagent acetone and filtered at 25° C. The filter cake was washed with 9 mL of reagent acetone and dried under vacuum overnight at 40° C. to give 1.52 g (31.2%) of crude ephedrine salt, mp 134-142° C.

Recrystallization of 1.32 g of the crude salt from 30 mL of hot reagent acetone gave 0.84 g (63.6% recovery) of purified ephedrine salt, mp 138-145° C.

The crude salt was converted to the corresponding acid by reaction with dilute hydrochloric acid. Assay of the resulting acids by chiral capillary electrophoresis (72/50 cm×50 μm uncoated capillary; 30° C., detection 195 nm; −25 kV; 25 mM phosphate pH 2.5+5 mM dimethyl B CD; Injection 0.5 sec×5″; samples at 0.2 mg/mL−10% acetonitrile) indicated that the enantiomeric excesses of the crude and purified salts were 89% and 99% ee, respectively.

### EXAMPLE 8

### Preparation of (1S,2R)-(+)-ephedrine Salt of (R)-(−)-2-(4-methoxyphenyl)-(4-nitroimidazolyl)propanoic Acid, Purification by Slurry in Ethyl Acetate

To a slurry of 10.0 g (34.33 mmol) of racemic 2-(4-methoxyphenyl)-(4-nitroimidazolyl)propanoic acid in 100 mL of ethyl acetate at 60° C. was added a solution of 6.0 g (34.43 mmol) of (1S,2R)-ephedrine hemihydrate dissolved in 30 mL of warm ethyl acetate followed by 20 mL of additional ethyl acetate. The resulting solution was reheated to 60° C. briefly, cooled to 55° C., and seeded with a crystal of desired product. After being allowed to cool to 25° C. and stirring overnight, the resulting slurry was filtered at 25° C. and the filter cake was washed with ethyl acetate (2×25 mL) and dried to give 6.74 g (43%) of crude product.

A sample of the crude product (0.75 g) was combined with 40 mL of methyl t-butyl ether and 30 mL of 1N HCl and the organic layer separated, washed with brine and dried with sodium sulfate. After filtration to remove sodium sulfate, the organic layer was concentrated to give 0.47 g of crude (R)-(−)-2-(4-methoxyphenyl)-(4-nitroimidazolyl)propanoic acid, which was 96.8% ee by chiral capillary electrophoresis.

The crude (1S,2R)-(+)-ephedrine salt of (R)-(−)-2-(4-methoxyphenyl)-(4-nitroimidazolyl)propanoic acid from above (5.0 g) was slurried in 75 mL of ethyl acetate and stirred at reflux for 1.5 h. After cooling, the resulting slurry was filtered and the filter cake was washed with ethyl acetate (2×25 mL) and dried to give 4.74 g of purified (1S,2R)-(+)-ephedrine salt of (R)-(−)-2-(4-methoxyphenyl)-(4-nitroimidazolyl)propanoic acid (94.8% recovery). A small sample was converted as above to (R)-(−)-2-(4-methoxyphenyl)-(4-nitroimidazolyl)propanoic acid which was 99.4% ee by chiral capillary electrophoresis.

### EXAMPLE 9

### Preparation of (1R,2S)-(−)-ephedrine Salt of (S)-(+)-2-(4-methoxyphenyl)-(4-nitroimidazolyl)propanoic Acid, Preparation by Recrystallization

Racemic 2-(4-methoxyphenyl)-(4-nitroimidazolyl)propanoic acid (1.02 g, 3.5 mmol) in 5 mL of acetone was combined with 0.58 g (3.5 mmol) of (1R,2S)-ephedrine and 1 mL of additional acetone at 25° C. The resulting solution was seeded with a crystal of (1R,2S)-(−)-ephedrine salt of (S)-(+)-2-(4-methoxyphenyl)-(4-nitroimidazolyl)propanoic acid and stirred at 25° C. overnight. The resulting slurry was filtered at 25° C. and the filter cake was washed with acetone (5 mL) and dried to give 0.55 g (34.3%) of crude product.

A small sample of the crude salt was converted to the free acid with 1 N HCl and extracted into ethyl acetate as above to give (S)-(+)-2-(4-methoxyphenyl)-(4-nitroimidazolyl)propanoic acid, which was 92.3% ee by chiral capillary electrophoresis.

A sample of the crude product was recrystallized from 11 mL of acetone to give the purified product, mp 139-147° C. Exact Mass 646.3509 (Calculated mass 646.3506 for C₃₈H₄₄N₇O₃). Specific rotation (methanol): [α]₅₈₉=+32.79°.

A small sample was converted to the free acid as in the examples above to give (S)-(+)-2-(4-methoxyphenyl)-(4-nitroimidazolyl)propanoic acid, 99.9% ee by chiral capillary electrophoresis.

### EXAMPLE 10

### Preparation of (1S,2R)-(+)-ephedrine Salt of (R)-(−)-2-(4-methoxyphenyl)-(4-nitroimidazolyl)propanoic Acid, Preparation by Slurry in Acetone/Water

A sample (0.50 g) of crude (1S,2R)-(+)-ephedrine salt of (R)-(−)-2-(4-methoxyphenyl)-(4-nitroimidazolyl)propanoic acid (96% ee) was suspended in 5 mL of 95% acetone/water and stirred at 25° C. for 3 h and then cooled to 0° C. and filtered. The resulting filter cake (0.40 g, 80% recovery) was converted to the corresponding acid by reaction with 30 mL of 1N HCl and extracted into 30 mL of ethyl acetate. The ethyl acetate layer was dried over sodium sulfate as above and concentrated to give 0.28 g of (R)-(−)-2-(4-methoxyphenyl)-(4-nitroimidazolyl)propanoic acid which was 98.1% ee by chiral capillary electrophoresis.

### EXAMPLE 11

### Preparation of (1S,2R)-(+)-ephedrine Salt of (R)-(−)-2-(4-methoxyphenyl)-(4-nitroimidazolyl)propanoic Acid, Preparation by Recrystallization from Acetonitrile

A sample (1.0 g) of crude (1S,2R)-(+)-ephedrine salt of (R)-(−)-2-(4-methoxyphenyl)-(4-nitroimidazolyl)propanoic acid (96% ee) was dissolved in 15 mL of acetonitrile at reflux, cooled to 25° C., and seeded with a crystal of desired product, and stirred at 25° C. overnight. An additional 15 mL of acetonitrile was added and the resulting slurry was cooled to O₂C, filtered, and washed with cold acetonitrile, and dried to give 0.77 g of crude (1S,2R)-(+)-ephedrine salt of (R)-(−)-2-(4-methoxyphenyl)-(4-nitroimidazolyl)propanoic acid.

The crude salt was converted to the corresponding acid by reaction with 1N HCl, extracted into ethyl acetate, and isolated as above to give 0.52 g of (R)-(−)-2-(4-methoxyphenyl)-(4-nitroimidazolyl)propanoic acid which was 99.5% ee by chiral capillary electrophoresis.

### EXAMPLE 12

### Preparation of (1S,2R)-(+)-ephedrine Salt of (R)-(−)-2-(4-methoxyphenyl)-(4-nitroimidazolyl)propanoic Acid, Preparation in 90% Ethyl Acetate/Acetone

(1S,2R)-(+)-ephedrine hemihydrate (0.60 g, 3.43 mmol) of 2-(4-methoxyphenyl)-(4-nitroimidazolyl)propanoic acid (1.0 g, 3.43 mmol) were combined in 20 mL of 90% (volume/volume) ethyl acetate/acetone and heated to refluxed. The mixture was cooled, and stirred at 25° C. overnight. The resulting slurry was filtered and the filter cake was washed with 90% ethyl acetate/acetone and dried to give 0.67 g (42.8%) of crude product.

A small sample of the crude salt was combined with 1 N HCl and the free acid was extracted into methyl t-butyl ether. Chiral capillary electrophoresis indicated that the acid was 90% ee.

### EXAMPLE 13

### Preparation of (1S,2R)-(+)-ephedrine Salt of (R)-(−)-2-(4-methoxyphenyl)-(4-nitroimidazolyl)propanoic Acid, Recrystallization from Acetone

(1S,2R)-(+)-ephedrine hemihydrate (11.97 g, 68.67 mmol) of 2-(4-methoxyphenyl)-(4-nitroimidazolyl)propanoic acid (20.0 g, 68.67 mmol) were combined in 275 mL of acetone and heated to reflux. The resulting solution was cooled to 0° C., and filtered. The filter cake was washed with cold ethyl acetate and dried to give 11.93 g (38%) of crude product. A small sample was converted to the free acid which was 96.8% ee by chiral capillary electrophoresis.

### EXAMPLE 14

### Preparation of (1S,2R)-(+)-ephedrine Salt of (R)-(−)-2-(4-methoxyphenyl)-(4-nitroimidazolyl)propanoic Acid, Recrystallization from Acetonitrile/Ethyl Acetate

(1S,2R)-(+)-ephedrine salt of (R)-(−)-2-(4-methoxyphenyl)-(4-nitroimidazolyl)propanoic acid (5.0 g, 97.4% ee) was slurried in 50 mL of acetonitrile and heated to 70° C. to give a clear solution. Ethyl acetate (50 mL) was added, maintaining 70° C. and the resulting solution was allowed to cool to 45° C. and seeded. After stirring overnight at 25° C., the resulting slurry was filtered and the filter cake was washed with ethyl acetate (2×25 mL) and dried to give 3.98 g (79.6% recovery) of purified salt. The purified salt (3.98 g) was slurried in 100 mL of methyl t-butyl ether and 50 mL of water and 12 mL of 1N HCl. The organic layer was separated and washed with brine and dried over sodium sulfate. After filtration to remove sodium sulfate, the filtrate was concentrated to give 2.47 g (77% from the crude salt) of (R)-(−)-2-(4-methoxyphenyl)-(4-nitroimidazolyl)propanoic acid which was >99.5% ee by chiral capillary electrophoresis.

### EXAMPLE 15

### Preparation of (1S,2R)-(+)-ephedrine Salt of (R)-(−)-2-(4-methoxyphenyl)-(4-nitroimidazolyl)propanoic Acid, Recrystallization from Acetonitrile/Ethyl Acetate

(1S,2R)-(+)-ephedrine salt of (R)-(−)-2-(4-methoxyphenyl)-(4-nitroimidazolyl)propanoic acid (5.0 g, composed of a mixture of several lots, 1.05 g, 98.6% ee, 1.13 g, 98.9% ee, 0.41 g, 94.7% ee, 0.96 g, 96.8% ee, and 1.45 g, 99.4% ee %) was slurried in 75 mL of ethyl acetate and 25 mL of acetonitrile and heated to 75° C. to give a clear solution. The resulting solution was allowed to cool to 50° C. and seeded. After stirring overnight, the resulting slurry was filtered at 25° C. and the filter cake was washed with ethyl acetate (2×25 mL) and dried to give 4.45 g (89% recovery) of purified salt. The purified salt (4.45 g) was slurried in 100 mL of methyl t-butyl ether, 50 mL of water, and 15 mL of 1N HCl. The organic layer was separated and washed with brine and dried over sodium sulfate. After filtration to remove sodium sulfate, the filtrate was concentrated to give 2.65 g (93.6% from purified salt) of (R)-(−)-2-(4-methoxyphenyl)-(4-nitroimidazolyl)propanoic acid which was >99.5% ee by chiral capillary electrophoresis.

We claim:

1. A method for obtaining a single enantiomer of nitroimidazolyl methoxyphenylpropionic acid of Formula A: 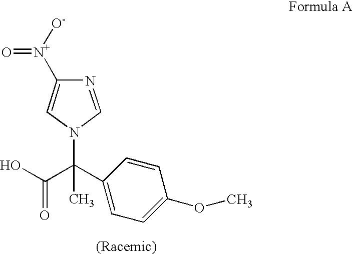
comprising the steps of:
(a) reacting a racemic mixture of a compound of Formula A with a resolving agent selected from the group consisting of levamisole, quinidine, brucine, (+)-cinchonine, (−)-cinchonidine, (1R,2S)-ephedrine and (1S,2R)-ephedrine in a solvent to produce a crystalline salt;
(b) isolating the crystalline salt; and
(c) optionally converting the crystalline salt to free acid thereby producing a substantially pure R or S enantiomer.

2. The method according to claim 1 wherein the solvent is selected from the group consisting of acetone, isopropyl alcohol, ethyl acetate/acetone, methyl tert-butyl ether/ethanol, ethyl acetate, isopropyl acetate, and methyl tert-butyl ether/methanol.

3. The method according to claim 1, wherein the isomer is a compound according to Formula B:

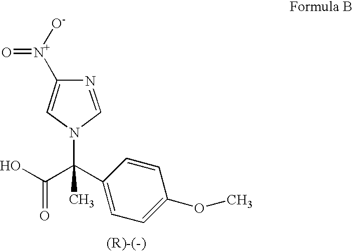

4. The method according to claim 1, wherein the isomer is a compound according to Formula C:

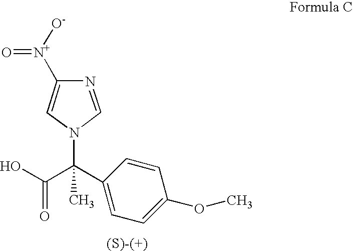

5. The method according to claim 3, wherein the compound of Formula B is a crystalline salt selected from the group consisting of levamisole salt, (−)-(1R,2S)-ephedrine salt, (+)-(1S,2R)-ephedrine salt, quinidine salt, brucine salt, (+)-cinchonine salt, (−) cinchonidine salt, and quinine salt.

6. The method according to claim 4, wherein the compound of Formula C is an organic salt selected from the group consisting of levamisole salt, (−)-(1R,2S)-ephedrine salt, (+)-(1S, 2R) ephedrine salt, quinidine salt, brucine salt, (+)-cinchonine salt, (−)-cinchonidine salt, and quinine salt.

7. An organic salt of the compound according to claim 3 selected from the group consisting of levamisole salt, (+)-(1S,2R) ephedrine salt, quinidine salt, brucine salt, (+)-cinchonine salt, (−)-cinchonidine salt, and quinine salt.

8. An organic salt of the compound according to claim 4 selected from the group consisting of, levamisole salt, (−)-(1R,2S)-ephedrine salt, quinidine salt, brucine salt, (+)-cinchonine salt, (−)-cinchonidine salt, and quinine salt.

9. In the process for preparing a compound of Formula X 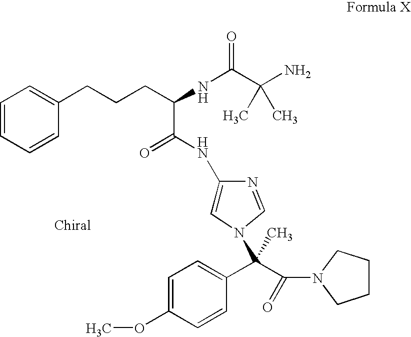
from an intermediate of Formula 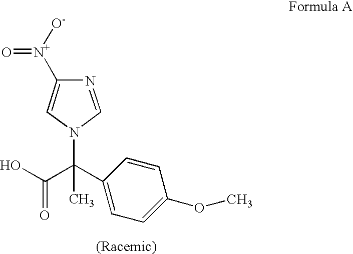
the improvement comprising resolution of the isomers according to claim 1.
